(19) United States
(12) Patent Application Publication
(10) Pub. No.: US 2025/0120990 A1
(43) Pub. Date: Apr. 17, 2025

(54) METHOD AND COMPOSITION FOR TREATING PARASITES IN AN ANIMAL

(71) Applicant: Quality Technology International, Inc., Elgin, IL (US)

(72) Inventors: Masahisa Ibuki, Tokyo (JP); Troy T. Lohrmann, McHenry, IL (US); Miloud Araba, Chapel Hill, NC (US); Jose Miguel Ruano, Frederica, DE (US)

(21) Appl. No.: 18/720,411

(22) PCT Filed: Dec. 14, 2022
(86) PCT No.: PCT/US2022/052873

§ 371 Date: Jun. 14, 2024

Related U.S. Application Data
(60) Provisional application No. 63/290,843, filed on Dec. 17, 2021.

(51) Int. Cl. A61K 31/7016; A23K 10/37; A23K 20/163; A23K 50/75; A61K 9/00; A61K 47/18; A61K 47/20; A61K 47/46; A61P 33/02

(52) U.S. Cl. CPC A61K 31/7016; A23K 10/37; A23K 20/163; A23K 50/75; A61K 9/0056; A61K 47/183; A61K 47/20; A61K 47/46; A61P 33/02

(57) ABSTRACT

The invention provides methods and compositions for treating a parasitic infection in an animal.

### FIELD

The present invention relates generally to methods and compositions for treating parasitic infections in animals.

### BACKGROUND

In the U.S., NAE (No Antibiotics Ever) producers cannot use ionophores for coccidiosis mitigation. Other common feed additives for coccidiosis mitigation in ABF (Antibiotic Free) production are chemicals (e.g., Zoamix®), coccidiosis vaccines, and plant extracts. While parasites can develop resistance to ionophore or chemical drugs over time, currently available live vaccines yield inconsistent results, and plant extracts and other mitigating alternatives also give inconsistent results (see, e.g., publication titled “A Survey of Sensitivity to Anticoccidial Drugs in 60 Isolates of Coccidia from broiler Chickens in Brazil and Argentina,” by Larry R. McDougald, Jose Maria Lamas Da Silva, Juan Solis, and Marucicio Braga, Avian Diseases, Vol. 31, No. 2, pp. 287-292 (1986), publication titled “Evaluating the Resistance of Eimeria Spp. Field Isolates to Anticoccidial Drugs Using Three Different Indices,” by F. Arabkhazaeli, M. Modrisanei, S. Nabian, B. Mansoori, and A. Madani, Iranian Journal of Parasitology, Vol. 8, No. 2, pp. 234-241 (2013), and publication titled “Anticoccidial Drug Resistance in Fowl Coccidia: The State of Play Revisited,” by R.Z. Abbas, Z. Iqbal, D. Blake, M.N. Khan, and M.K. Saleemi, World's Poultry Science Journal, Volume 67, Issue 2, pp. 337-350 (2011)).

Currently there is a need for agents that are useful for treating parasitic infections in animals, such as, for example, coccidiosis in chickens, which causes bird performance and health impairment associated with significant economic loss (see, e.g., publication titled “Re-calculating the Cost of Coccidiosis in Chickens,” by Damer P. Blake, Jolene Knox, Ben Dehaeck, Ben Huntington, Thilak Rathinam, Venu Ravipati, Simeon Ayoade, Will Gilbert, Ayotunde O. Adebambo, Isa Danladi Jatau, Muthusamy Raman, Daniel Parker, Jonathan Rushton, and Fiona M. Tomley, Veterinary Research, Vol. 51, Article Number 115 (2020), and publication titled “High Cost of Coccidiosis in Broilers,” by The Poultry Site, www.thepoultrysite.com/news/2013/02/high-cost-of-coccidiosis-in-broilers (2013)).

### SUMMARY

In one aspect the present invention provides a method that is useful for treating a parasitic infection in an animal. Accordingly, the invention provides a method comprising, treating an infection of a parasite in an animal (e.g., decreasing the cycling level of the parasite), by administering β-1,4-mannobiose (mannanase-hydrolyzed copra meal) to the animal.

In another embodiment, the invention provides a composition for treating an infection of a parasite in an animal comprising an effective amount of β-1,4-mannobiose.

In another embodiment, the invention provides β-1,4-mannobiose for the prophylactic or therapeutic treatment of a parasitic infection.

In another embodiment, the invention provides β-1,4-mannobiose for the prophylactic or therapeutic treatment of a parasitic infection in combination with animal feed.

In another embodiment, the invention provides β-1,4-mannobiose for the prophylactic or therapeutic treatment of a parasitic infection in combination with animal feed, wherein the amount of β-1,4-mannobiose is 0.000375% to 0.075% by weight of the dry matter portion of the feed.

In another aspect of the present invention, there is provided a composition comprising an effective amount of β-1,4-mannobiose for use in the treatment and/or prophylaxis of an infection of a parasite in an animal.

In embodiments, the composition further comprises animal feed. In embodiments, the β-1,4-Mannobiose is provided as mannanase-hydrolyzed copra meal.

In embodiments, the β-1,4-mannobiose is present in the composition in the amount of about 0.000375% to 0.075% by weight of the dry matter portion of the feed. In embodiments, the β-1,4-mannobiose is present in the composition in the amount of about 0.005% by weight of the dry matter portion of the feed.

In embodiments, the composition further comprises a compound selected from the group consisting of essential oils, plant extracts, probiotics, postbiotics, prebiotics, antibiotics, anthelmintics, and antibiotics.

In embodiments, the parasite is a protozoa. In embodiments, the parasite is a protozoa of the phylum Apicomplexa, family Eimeriidae. In embodiments, the parasite belongs to the genus Eimeria. In embodiments, the parasite is E. maxima (Eimeria maxima).

In embodiments, the infection of a parasite is coccidiosis.

In embodiments, the animal is a cow, a horse, a pig, a sheep, a chicken, a turkey, a duck, or a goose. In another embodiment, the animal is a non-human animal.

In embodiments, an effective amount of β-1,4-mannobiose is an amount that inhibits the parasite, eliminates the parasite, relieves one or more symptoms caused by the parasite and/or kills some or all of the parasite.

In another aspect of the present invention, there is provided β-1,4-mannobiose for use in the treatment and/or prophylaxis of a parasitic infection.

In embodiments, the β-1,4-mannobiose is combined with animal feed. In embodiments, the β-1,4-Mannobiose is provided as mannanase-hydrolyzed copra meal.

In embodiments, the β-1,4-mannobiose is present in the amount of about 0.000375% to 0.075% by weight of the dry matter portion of the feed. In embodiments, the β-1,4-mannobiose is present in the amount of about 0.00075% to 0.037% by weight of the dry matter portion of the feed. In embodiments, the β-1,4-mannobiose is present in the amount of about 0.005% by weight of the dry matter portion of the feed.

In embodiments, the β-1,4-Mannobiose is provided as mannanase-hydrolyzed copra meal (MCM), the MCM is provided to the animal as part of the animal's feed, and the feed comprises 0.0125% to 0.5% MCM by weight. In embodiments, the feed comprises 0.025% to 0.25% MCM by weight. In embodiments, the feed comprises about 0.05% MCM by weight.

In embodiments, the β-1,4-mannobiose is combined with a compound selected from the group consisting of essential oils, plant extracts, probiotics, postbiotics, prebiotics, antibiotics, anthelmintics, and antibiotics.

In embodiments, the parasite is a protozoa. In embodiments, the parasite is a protozoa of the phylum Apicomplexa, family Eimeriidae. In embodiments, the parasite belongs to the genus Eimeria. In embodiments, the parasite is E. maxima.

In embodiments, the parasitic infection is coccidiosis.

In embodiments, the animal is a cow, horse, pig, sheep, chicken, turkey, duck, or goose.

### BRIEF DESCRIPTION OF THE DRAWINGS

FIG. 1 is a chart 101 showing the Adj. (adjusted) FCR (feed conversion ratio) at fourteen (14) days associated with the treatments in Example 2.

FIG. 2 is a chart 201 showing the weight gain at fourteen (14) days associated with the treatments in Example 2.

FIG. 3 is a chart 301 showing mortality due to E. maxima infection at fourteen (14) days associated with the treatments in Example 2.

FIG. 4 is a chart 401 showing the feed intake (kilograms per cage) at fourteen (14) days associated with the treatments in Example 2.

FIG. 5 is a chart 501 showing the oocysts per gram (OPG) at seven (7) days associated with the treatments in Example 2.

FIG. 6 is a chart 601 showing the Adj. (adjusted) FCR (feed conversion ratio) at seven (7) days associated with the treatments in Example 2.

FIG. 7 is a chart 701 showing the weight gain at seven (7) days associated with the treatments in Example 2.

FIG. 8 is a chart 801 showing the feed intake (kilograms per cage) at seven (7) days associated with the treatments in Example 2.

### DETAILED DESCRIPTION

Although the following detailed description contains many specifics for the purpose of illustration, a person of ordinary skill in the art will appreciate that many variations and alterations to the following details are within the scope of the invention. Specific examples are used to illustrate particular embodiments; however, the invention described in the claims is not intended to be limited to only these examples, but rather includes the full scope of the attached claims. Accordingly, the following preferred embodiments of the invention are set forth without any loss of generality to, and without imposing limitations upon the claimed invention. Further, in the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

It is specifically contemplated that the present invention includes embodiments having combinations and subcombinations of the various embodiments and features that are individually described herein (i.e., rather than listing every combinatorial of the elements, this specification includes descriptions of representative embodiments and contemplates embodiments that include some of the features from one embodiment combined with some of the features of another embodiment, including embodiments that include some of the features from one embodiment combined with some of the features of embodiments described in the patents and application publications incorporated by reference in the present application). Further, some embodiments include fewer than all the components described as part of any one of the embodiments described herein.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

Certain marks referenced herein may be common-law or registered trademarks of third parties affiliated or unaffiliated with the applicant or the assignee. Use of these marks is for providing an enabling disclosure by way of example and shall not be construed to limit the scope of the claimed subject matter to material associated with such marks.

The following definitions are used, unless otherwise described.

The term “parasitic” includes organisms that live on or inside another organism and benefit at the other organism's expense. In one embodiment, the parasite is a protozoa (e.g., giardia, malaria, or coccidia) or a worm (e.g., a hookworm. tapeworm, or fluke). In one embodiment, the parasitic infection is caused by a protozoa of the phylum Apicomplexa, family Eimeriidae. In one embodiment, the parasitic infection is caused by a species belong to the genus Eimeria. In one embodiment, the parasitic infection is caused by the species E. maxima.

The term “treating” includes inhibiting the parasite, eliminating the parasite. and/or relieving one or more symptoms caused by the parasite. The term “treating” also refer to both therapeutic treatment and/or prophylactic treatment or preventative measures, wherein the object is to prevent or slow down (lessen) an undesired physiological change or disorder, such as, for example, a parasitic infection. For example, beneficial or desired results include, but are not limited to, alleviation of symptoms, diminishment of extent of a parasitic infection, stabilized (i.e., not worsening) state of a parasitic infection, delay or slowing of progression of a parasitic infection, amelioration or palliation of a parasitic infection, and remission (whether partial or total), whether detectable or undetectable. “Treating,” can also mean prolonging survival as compared to expected survival if not receiving treatment. Animals in need of treatment include those already with the parasitic infection as well as those prone to have the parasitic infection or those in which the parasitic infection is to be prevented. In one embodiment “treating” does not include preventing or prevention. In one embodiment “treating” does include preventing or prevention.

The phrase “effective amount” includes but is not limited to an amount that (i) treats or prevents a parasitic infection, (ii) attenuates, ameliorates, or eliminates one or more symptoms of the parasitic infection, or (iii) prevents or delays the onset of one or more symptoms of the parasitic infection. In one embodiment, the β-1,4-mannobiose is provided to the animal as part of the animal's feed. In one embodiment, the feed comprises 0.000375% to 0.075% β-1,4-mannobiose by weight. In one embodiment, the feed comprises 0.00075% to 0.037% 1,4 β-1,4-mannobiose by weight. In one embodiment, the feed comprises about 0.005% β-1,4-mannobiose by weight.

The term “animal” as used herein refers to humans, higher non-human primates, rodents, cows, horses, pigs, sheep, dogs, cats, poultry, crustaceans, and fish. In one embodiment, the animal is a cow, horse, pig, or sheep. In another embodiment, the animal is a fish. In another embodiment, the animal is a crustacean such as shrimp. In another embodiment, the animal is poultry (e.g., a chicken, turkey, duck, or goose). In another embodiment, the animal is a chicken. In another embodiment, the animal is a non-human animal. In one embodiment, the animal (e.g., the chicken) is not infected by salmonella.

β-1,4-mannobiose as well as animal feeds that comprise β-1,4-mannobiose can be prepared as described in U.S. Pat. No. 8,999,374.
Copra is a dried coconut fruit. It is grayish white and contains about 40-65% of good quality fat. It is mainly produced in Southeast Asian countries and Pacific Islands. Pressed copra oil is used as a raw oil and fat for processed foods such as margarine, and it is also used as a raw material for daily industrial products such as soap and candles because it is less aggressive to human body. Copra oil squeeze (by-product) is called copra meal (copra flake), and it becomes organic fertilizer and livestock feed. Being rich in vitamins, oils and fats, it is also known as a fattening feed for branded beef and pork in Japan. In one embodiment, β-1,4-mannobiose can be administered as mannanase-hydrolyzed copra meal (MCM). In one embodiment, the mannanase-hydrolyzed copra meal is provided to the animal as part of the animal's feed. In one embodiment, the feed comprises 0.0125% to 0.5% mannanase-hydrolyzed copra meal by weight. In one embodiment, the feed comprises 0.025% to 0.25% mannanase-hydrolyzed copra meal by weight. In one embodiment, the feed comprises about 0.05% mannanase-hydrolyzed copra meal by weight. In one embodiment, the mannanase-hydrolyzed copra meal can be prepared as described in Example 4.

The invention will now be illustrated by the following non-limiting Examples.

### EXAMPLES

### Example 1. Anticoccidial Trial

The anticoccidial efficacy of β-1,4-mannobiose was evaluated in broiler chickens in a rapid 14-day E. maxima challenge model. The model was carried out using conditions like those described by Guidance #217, “Evaluating the Effectiveness of Anticoccidial Drugs in Food-Producing Animals,” U.S. Department of Health and Human Services, Food and Drug Administration, Center for Veterinary Medicine (2012). The efficacy of β-1,4-mannobiose (in the form of MCM) was compared with the efficacy of IMW50®, B. coagulans (Bacillus coagulans), and Zoamix® (a commercially used coccidiostat).

The study involved:
a 14-day cage battery trial using male Cobb 500 broiler chickens;
4 cages/treatment, 10 birds/cage;
Except birds in Treatment 1, all other chicks were gavaged (vaccinated) at 1-day of age with 1,200 oocysts of E. maxima (equivalent to a 3X cocci vaccine dose);
All test materials were delivered through the feed (mash);
Diets and water were provided ad lib throughout trial;
OPG (Oocysts per gram) counts were taken at peak of E. maxima cycling—7 days post infection (8^{th} day actually);
Feed and bird weights were measured at 7 and 14 days of age;
Birds received routine vaccination (HVT, SB1) on 1 day of age; no cocci vaccine; and
fecal OPG's, feed-to-grain ratio F/G, body weight gain (BWG), and mortality parameters were measured.

TABLE 1
Results at Day 14 of Example 1

| | Feed | | Wt. Gain | E. maxima |
|---|---|---|---|---|
| Treatment | Intake | Adj. | (Kg/ | Mortality |
| | (Kg/Cage) | FCR | Bird) | (%) |
| 1. No additive, No | 3.074ª | 1.327^{d} | 0.247^{abc} | 0.0ª |
| E. maxima challenge | | | | |
| 2. No additive + | 3.164ª | 1.610ª | 0.207^{c} | 7.5ª |
| E. maxima challenge | | | | |
| 3. Mico-Aid (4x dose) | 3.131ª | 1.523ª^{b} | 0.232^{abc} | 12.5ª |
| 4. Orego-Stim (1.6x dose) | 3.271ª | 1.490^{bc} | 0.234^{abc} | 2.5ª |
| 5. Magni-Phi (2x dose) | 3.069ª | 1.533^{ab} | 0.222^{bc} | 7.5ª |
| 6. Zoamix (1x dose | 3.464ª | 1.360^{d} | 0.265^{ab} | 0.0ª |
| or 500 g/ton) | | | | |
| 7. Plant Polyphenol | 3.692ª | 1.562ª^{b} | 0.268ª | 7.5ª |
| blend (500 g/ton) | | | | |
| 8. IMW50 (1 lb/ton) | 3.427ª | 1.375^{cd} | 0.255^{ªb} | 2.5ª |
| 9. MCM (1 lb/ton) | 3.120ª | 1.390^{cd} | 0.241^{abc} | 5.0ª |
| 10. B. coagulans | 3.082ª | 1.483^{bc} | 0.230^{abc} | 7.5ª |
| (200,000 cfu/g) | | | | |

Micro-Aid ®, Orego-Stim ™ and Magni-Phi ® were commercially sourced. IMW50 ® and B. coagulans are available from Quality Technology International, Inc. (QTI, 1707 N. Randall Road, Suite 300, Elgin, Illinois 60123).

Results are summarized below.
Birds gavaged with cocci vaccine and given no feed additives had the worst F/G and BWG birds fed diets supplemented with Zoamix® doing very well.
Birds given 4× dose of Micro-Aid® performed poorly and showed minimal cocci mitigating properties. Birds fed Plant Polyphenol blend, Magni-Phi®, and Orego-Stim™ performed slightly better than Micro-Aid® and appear to have some mitigation on cocci cycling.
Birds fed diets supplemented with IMW50® or MCM performed equally to non-infected control birds and demonstrated good cocci mitigating properties.
At 200K cfu/g, B. coagulans added to the diet also appears to have effects on cocci cycling.

### Example 2. Anticoccidial Trial

The anticoccidial efficacy of β-1,4-mannobiose was evaluated in broiler chickens in a rapid 14-day E. maxima challenge model. The model was carried out using conditions similar to those described by Guidance #217, “Evaluating the Effectiveness of Anticoccidial Drugs in Food-Producing Animals,” U.S. Department of Health and Human Services, Food and Drug Administration, Center for Veterinary Medicine (2012). The efficacy of β-1,4-mannobiose (in the form of MCM) was compared with the efficacy of IMW50®, B. coagulans, and Zoamix® (an FDA-approved, commercial coccidiostat).

The study involved:
A 14-day cage battery trial using male Cobb 500 broiler chickens;
10 treatments, 4 cages/treatment, 10 birds/cage;
except birds in Treatment 1. all other chicks were gavaged (vaccinated) at 1-day of age with 1,200 oocysts of E. maxima (equivalent to a 3X cocci vaccine dose).
All test materials were delivered through the feed (mash);
Diets and water were provided ad lib throughout trial;
OPG counts were taken at peak of E. maxima cycling—7 days post infection (8^{th} day actually);
Feed and bird weights were measured at 7 and 14 days of age;
Birds received routine vaccination (HVT, SB1) on 1 day of age; No cocci vaccine; and
Fecal OPG's, F/G, BWG, and mortality parameters were measured.

TABLE 2
Treatment Descriptions for Example 2
E. Maxima Anticoccidial Trial

| Treatment | | # Cages per | # Birds/ | Feed Inclusion |
|---|---|---|---|---|
| # | Description | Treatment | Cage | rate |
| 1 | Positive Control, No EM | 4 | 10 | None |
| 2 | Negative Control, EM | 4 | 10 | None |
| 3 | Zoamix | 4 | 10 | 500 g/ton |
| 4 | IMW50 | 4 | 10 | 500 g/ton |
| 5 | MCM | 4 | 10 | 500 g/ton |
| 6 | ½ IMW50 + ½ MCM | 4 | 10 | (250 g + 250 g)/ton |
| 7 | B. coagulans | 4 | 10 | 300K cfu/g |
| 8 | IMW50 + B. coagulans | 4 | 10 | 250 g/ton + |
| | | | | 300K cfu/g |
| 9 | MCM + B. coagulans | 4 | 10 | 250 g/ton + |
| | | | | 300K cfu/g |
| 10 | IMW50 + Betaine | 4 | 10 | (500 g +100 g)/ton |

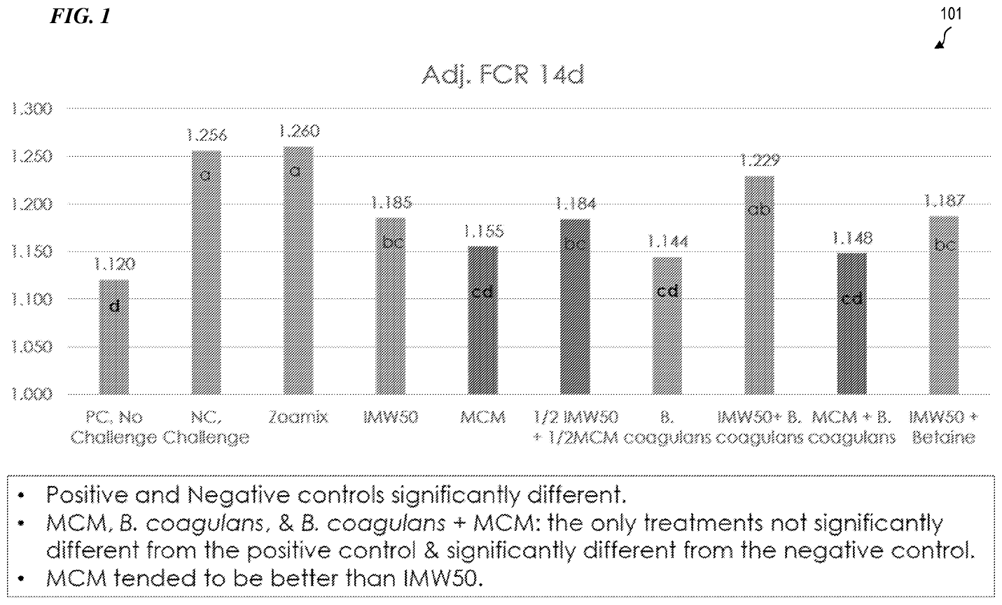

Results are shown in FIGS. 1-8 and are summarized below.
Birds challenged with E. maxima cocci vaccine and given no feed additives had the worst feed conversion;
MCM, IMW50®, B. coagulans showed anti-coccidial effect;
MCM, B. coagulans tended to show best anticoccidial efficacy;
No interactions were apparent when 2 test materials were combined under the trial's design; and Zoamix®, the industry's standard coccidiostat, performed poorly in this trial.

FIG. 1 is a chart 101 showing the Adj. (adjusted) FCR (feed conversion ratio) at fourteen (14) days associated with the treatments in Example 2.

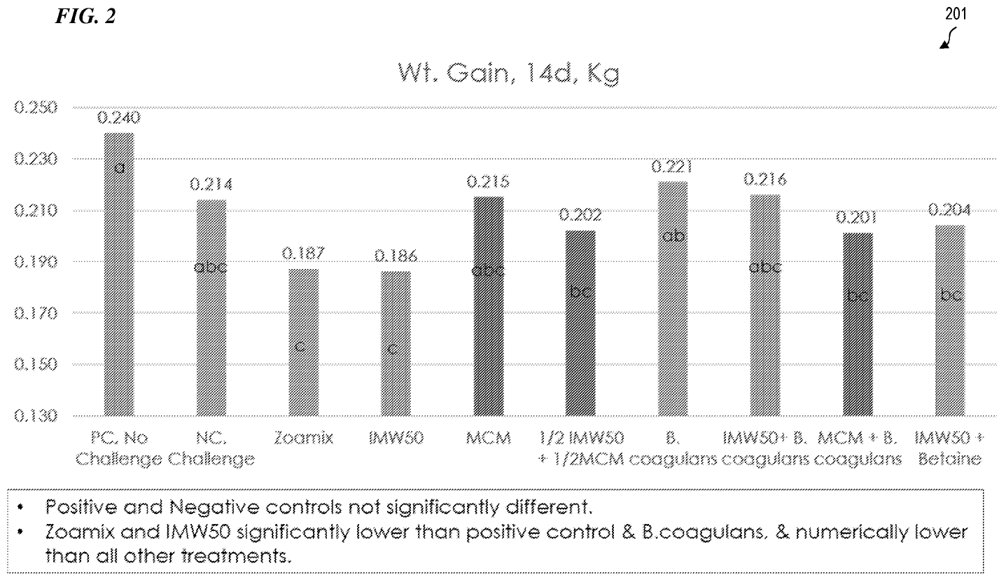

FIG. 2 is a chart 201 showing the weight gain at fourteen (14) days associated with the treatments in Example 2.

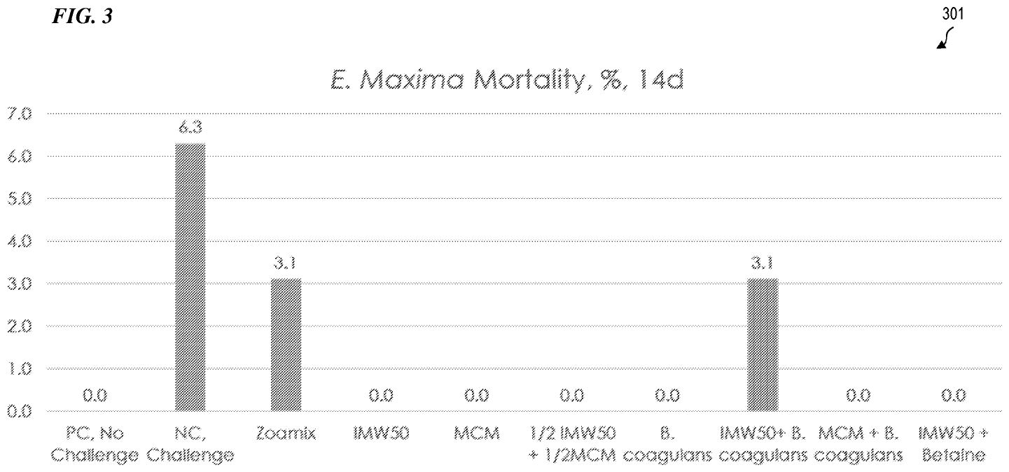

FIG. 3 is a chart 301 showing mortality due to E. maxima infection at fourteen (14) days associated with the treatments in Example 2.

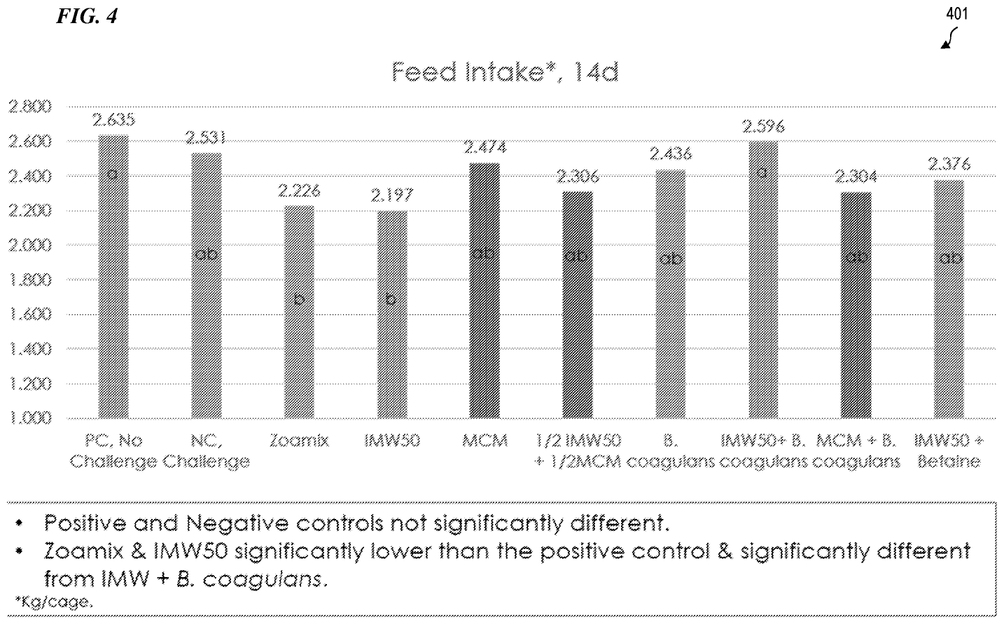

FIG. 4 is a chart 401 showing the feed intake (kilograms per cage) at fourteen (14) days associated with the treatments in Example 2.

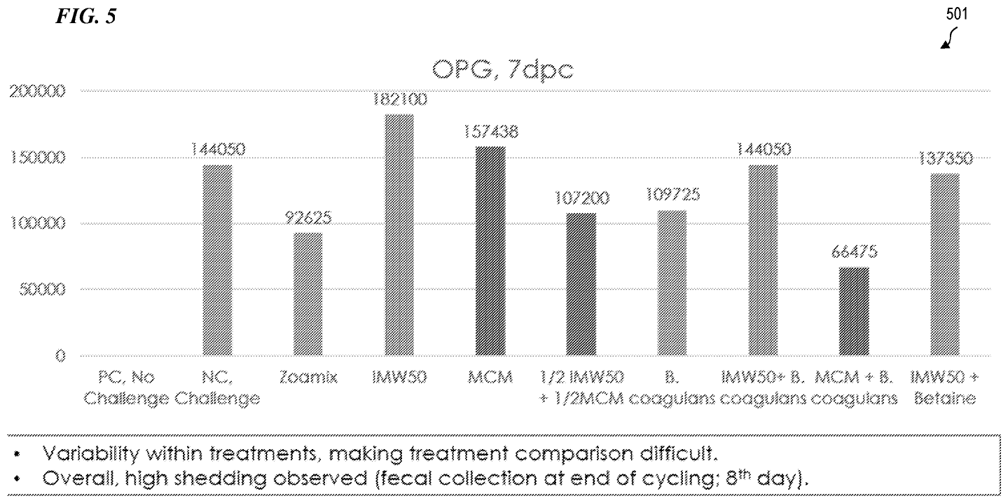

FIG. 5 is a chart 501 showing the oocysts per gram (OPG) at seven (7) days associated with the treatments in Example 2.

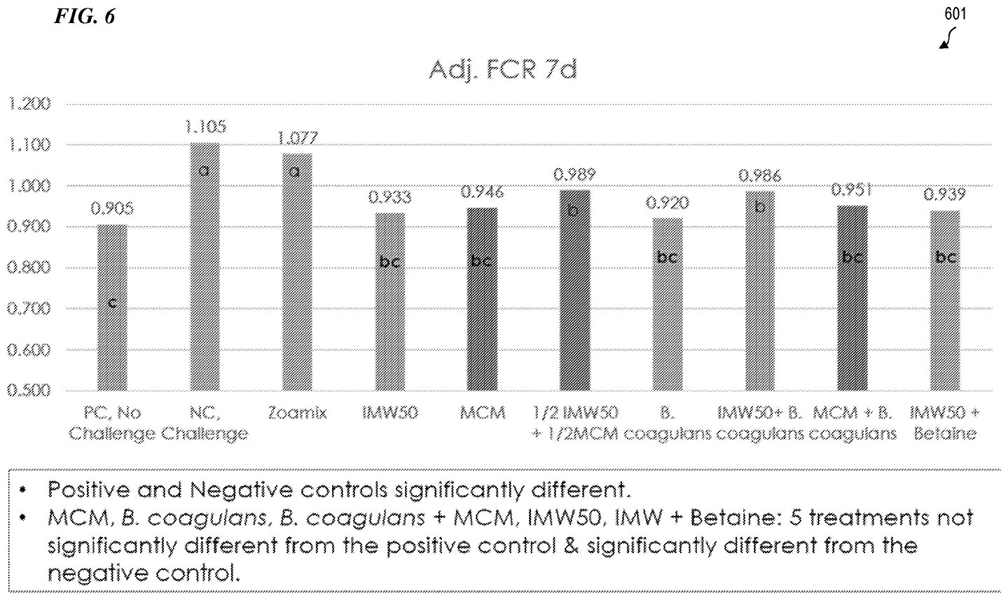

FIG. 6 is a chart 601 showing the Adj. (adjusted) FCR (feed conversion ratio) at seven (7) days associated with the treatments in Example 2.

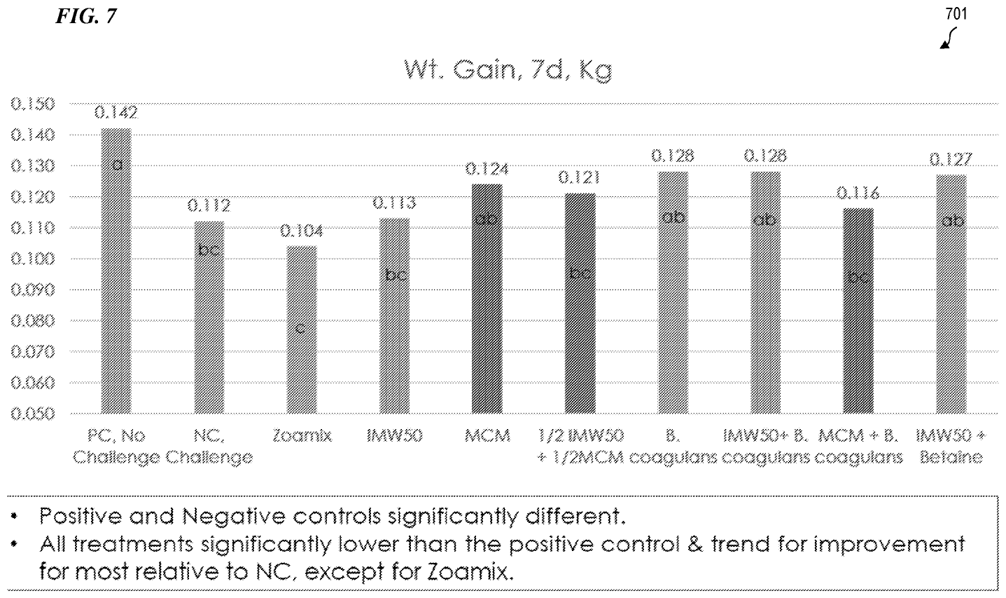

FIG. 7 is a chart 701 showing the weight gain at seven (7) days associated with the treatments in Example 2.

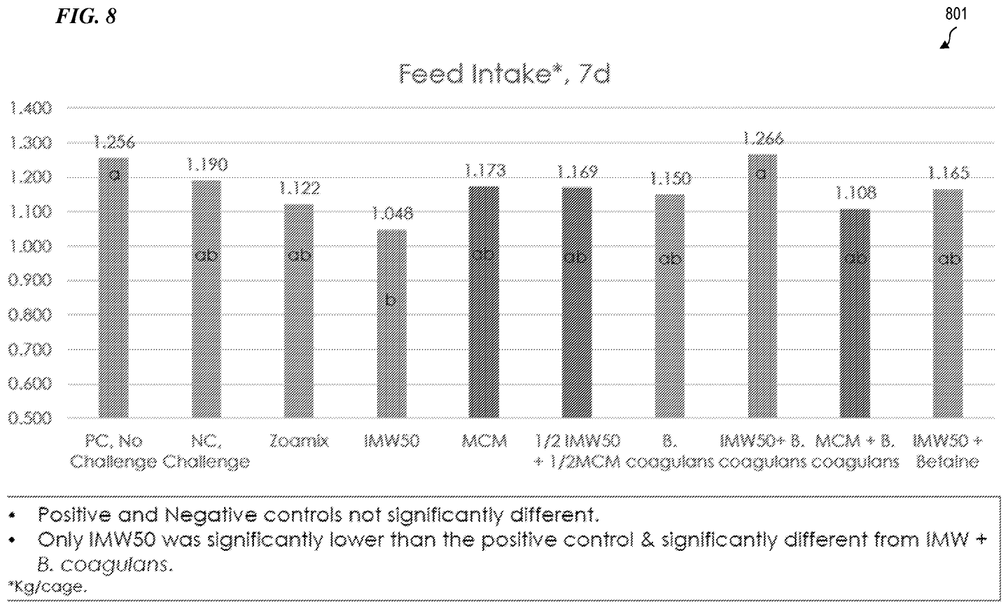

FIG. 8 is a chart 801 showing the feed intake (kilograms per cage) at seven (7) days associated with the treatments in Example 2.

### Example 3. An Example Feed Composition Containing MCM

TABLE 3
Example MCM Feed Composition for 1 Metric Ton of Feed

| Ingredient | | | |
|---|---|---|---|
| Code | Ingredient Name | Kg | % |
| | MCM | 0.5 | 0.05 |
| 22 | Corn, yellow, grain | 559.88 | 55.94 |
| 101 | Soybean meal dehulled, solvent | 393.11 | 39.31 |
| 41 | Fat, vegetable | 16.31 | 1.63 |
| 152 | Calcium carbonate | 10.48 | 1.05 |
| 156 | Dicalcium phosphate | 9.74 | 0.97 |
| 220 | Methionine MHA | 3.24 | 0.32 |
| 162 | SALT, PLAIN (NaCl) | 2.88 | 0.29 |
| 200 | L-LYSINE | 1.82 | 0.18 |
| 204 | Trace Mineral | 0.75 | 0.08 |
| 202 | Vitamin premix | 0.65 | 0.07 |
| 206 | L-Threonine 98.5 | 0.52 | 0.05 |
| 223 | quantum blu | 0.11 | 0.01 |

The example feed composition shown in Table 3 contains 0.5 kilogram (kg) of MCM, but the amount of MCM in the example feed composition can vary in a range of about 0.125 kg to about 5 kg with the amount of MCM being added at the expense of one or more other ingredients (e.g., if MCM is added at the expense of corn and 5 kg of MCM is added instead of 0.5 kg of MCM, then 555.38 kg of corn would be used instead of 559.88 kg of corn).

### Example 4—Preparation of Mannanase-hydrolyzed Copra Meal (MCM)

As described in U.S. Pat. No. 8,999,374, the following process can be used to prepare mannanase-hydrolyzed copra meal: 150 parts of enzymic solution, in which 0.25 parts of enzyme Hemicellulase GM “AMANO” (Amano Pharmaceutical Co., Ltd.) was dissolved, was functioned to 100 parts of copra meal containing mannan at 30% and water at 4.2% for 12 hours at 60° C., and then the solution was dried with a fluidized bed dryer until its water content reduced to 9.3% to yield 106 parts of dry powder. When the mannose content and β-1,4-mannobiose content in this dry powder were measured, it was found that 1.36 parts of mannose and 12.35 parts of β-1,4-mannobiose (41.2% based on mannan; 12.9% in terms of dry matter) were produced.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method comprising, treating an infection of a parasite in an animal, by administering an effective amount of β-1,4-mannobiose to the animal, wherein the animal is a chicken, turkey, duck, goose, cow, horse, pig, or sheep.

2. The method of claim 1, wherein the parasite is a protozoa or a worm.

3. The method of claim 1, wherein the parasite is a protozoa.

4. The method of claim 1, wherein the parasite is a protozoa of the phylum Apicomplexa, family Eimeriidae.

5. The method of claim 1, wherein the parasite belongs to the genus Eimeria.

6. The method of claim 1, wherein the parasite is E. maxima.

7. The method of claim 1 or claim 2 or claim 3 or claim 4 or claim 5 or claim 6, wherein the animal is a cow, horse, pig, or sheep.

8. The method of claim 1 or claim 2 or claim 3 or claim 4 or claim 5 or claim 6, wherein the animal is a chicken, turkey, duck, or goose.

9. The method of claim 1 or claim 2 or claim 3 or claim 4 or claim 5 or claim 6, wherein the animal is a chicken.

10. The method of claim 8, wherein a composition comprising at least about 0.000375% β-1,4-mannobiose by weight is administered to the animal.

11. The method of claim 10, wherein the composition comprises animal feed.

12. The method of claim 11, wherein the amount of β-1,4-mannobiose is about 0.005% of the dry matter portion of the feed by weight.

13. The method of claim 10, wherein the amount of β-1,4-mannobiose administered to the animal is effective to inhibit the parasite, eliminate the parasite, and/or relieve one or more symptoms caused by the parasite.

14. The method of claim 10, wherein the amount of β-1,4-mannobiose administered to the animal is effective to kill some or all of the parasite in the animal.

15. The method of claim 10, wherein the animal is not infected with salmonella.

16. The method of claim 10, further comprising administering another anti-parasitic compound to the animal.

17. The method of claim 16, wherein the other anti-parasitic compound is selected from the group consisting of essential oils, plant extracts, probiotics, postbiotics, prebiotics, antibiotics, anthelmintics, and antibiotics.

18.-32. (canceled)
